(19) United States
(12) Patent Application Publication
(10) Pub. No.: US 2018/0310603 A1
(43) Pub. Date: Nov. 1, 2018

(54) BEVERAGE FOR PREOPERATIVE USE

(71) Applicant: Surgistrong, Mandeville, LA (US)

(72) Inventors: William Charles Conway, Santa Barbara, CA (US); Siobhan Trotter, Mandeville, LA (US)

(21) Appl. No.: 15/962,954

(22) Filed: Apr. 25, 2018

Related U.S. Application Data
(60) Provisional application No. 62/491,004, filed on Apr. 27, 2017.

(51) Int. Cl. A23L 33/105; A61K 9/00; A61K 31/718; A61K 31/198; A61K 31/07; A61K 31/593; A61K 31/522; A23L 33/125; A23L 33/155; A23L 33/175; A23L 2/60

(52) U.S. Cl. CPC A23L 33/105; A61K 9/0053; A61K 31/718; A61K 31/198; A61K 31/07; A23V 2002/00; A61K 31/522; A23L 33/125; A23L 33/155; A23L 33/175; A23L 2/60; A61K 31/593

(57) ABSTRACT

Disclosed herein is a preoperative beverage that is formulated to be administered prior to an operation or surgical procedure. In particular, the preoperative beverage is a water-based beverage that is configured to provide a patient with calories and nutrients while also maintaining an osmolality that is suitable for preoperative use The preoperative beverage may have a pH greater than approximately 4.3, one or more carbohydrates to provide at least 200 calories, and an osmolality ranging between 250-300 mOsm to facilitate absorption in less than two hours. The preoperative beverage may also include one or more amino acids, as well as other nutritional supplements to facilitate the health and comfort of the patient before and after the operation or surgical procedure.

### CROSS-REFERENCE TO RELATED APPLICATION

This application is a non-provisional patent application of and claims the benefit to U.S. Provisional Patent Application No. 62/491,004, filed Apr. 27, 2017, and titled “Beverage for Preoperative Use,” the disclosure of which is hereby incorporated herein by reference in its entirety.

### FIELD

The described embodiments relate generally to a beverage suitable for consumption before a surgical procedure or operation. More particularly, the present embodiments relate to a water-based preoperative beverage that is configured to provide a patient with calories and nutrients while also maintaining an osmolality that is suitable for preoperative use.

### BACKGROUND

To avoid complications during a surgical procedure or operation in which anesthesia is administered, traditional preoperative guidelines may include a fasting period beginning the night before surgery. Under these guidelines, a patient may be asked to refrain from ingesting any solid foods or non-clear fluids for the fasting period. By fasting before surgery, the patient may experience a reduced risk of perioperative pulmonary aspiration or other related complications.

However, subjecting the patient to an extended fasting period before an operation may prevent the patient from receiving calories and/or nutrients that may be helpful during postoperative recovery. Disclosed herein is a beverage that may be administered to patients before a surgical procedure to improve the patient's comfort and facilitate postoperative recovery.

### SUMMARY

Some example embodiments are directed to a method of administering a preoperative beverage to a patient. The method may include fasting from solid foods for a fasting period of at least 6 hours. The method may also include administering the preoperative beverage during the fasting period and at least 2 hours before a surgical procedure. The preoperative beverage may include a water-based solution having a pH of 4.5 or greater. The preoperative beverage may include a specialty water including, for example, hydrogen-rich water, H₂ infused water, alkaline water, and other types of specially formulated water.

The preoperative beverage may also include a carbohydrate including maltodextrin in quantity ranging between 10-15% of the preoperative beverage by weight and an amino acid in a quantity ranging between 0.1 and 0.3% of the preoperative beverage by weight. The preoperative beverage may have an osmolality ranging between 100-300 mOsm. In some implementations, the preoperative beverage may have an osmolality ranging between 250-300 mOsm. In some examples, the preoperative beverage is configured to be absorbed by a patient's stomach within approximately 90 minutes from the time of ingestion.

In some embodiments, the preoperative beverage is shelf stable for a period of at least 12 months when packaged. The preoperative beverage may be aseptically packaged in an aseptic container that is configured to maintain or increase the shelf life of the preoperative beverage. In some embodiments, the preoperative beverage is packaged using a hot-fill or heat-pasteurizing process.

In some embodiments, the preoperative beverage includes vitamin A in a quantity ranging between 0.0005 and 0.002% of the preoperative beverage by weight. In some embodiments, the preoperative beverage includes vitamin D3 in a quantity ranging between 0.0005 and 0.002% of the preoperative beverage by weight. In some embodiments, the preoperative beverage includes caffeine in a quantity ranging between 0.005 and 0.05% of the preoperative beverage by weight. In some embodiments, the preoperative beverage includes a natural sweetener including monk fruit extract in a quantity ranging between 0.005 and 0.05% by weight. In some embodiments, the preoperative beverage includes one or more antioxidants including, for example, resveratrol, lactulose, or epicatechins.

Some example embodiments are directed to a preoperative beverage for administration between 2 to 6 hours before a surgical procedure. The preoperative beverage may include a water-based solution having a pH of 3.5 or greater. The preoperative beverage may include a water-based solution having a pH of 4 or greater. In some cases, the preoperative beverage may include a water-based solution having a pH of 4.5 or greater. In some cases, the preoperative beverage may include a water-based solution having a pH of 4.8 or greater. The preoperative beverage may also include a carbohydrate including maltodextrin in a quantity ranging between 10-15% of the preoperative beverage by weight. The preoperative beverage may also include an acid including L-citruline in a quantity ranging between 0.1 and 0.3% of the preoperative beverage by weight. In some implementations, the preoperative beverage has an osmolality ranging between 100-300 mOsm. In some implementations, the preoperative beverage has an osmolality ranging between 250-300 mOsm.

In some embodiments, the preoperative beverage is shelf stable for a period of at least 12 months when packaged. The preoperative beverage may be packaged using an aseptic container that may help maintain or increase the shelf life of the preoperative beverage. In some embodiments, the preoperative beverage is packaged using a hot-fill or heat-pasteurizing process.

In some embodiments, the preoperative beverage includes caffeine in a quantity ranging between 0.005 and 0.05% of the preoperative beverage by weight and a natural sweetener including monk fruit extract in a quantity ranging between 0.005 and 0.05% by weight. In some embodiments, the preoperative beverage includes vitamin A in a quantity ranging between 0.0005 and 0.002% of the preoperative beverage by weight and vitamin D3 in a quantity ranging between 0.0005 and 0.002% of the preoperative beverage by weight.

Some example embodiments are directed to a method of administering a preoperative beverage including: abstaining from solid foods and non-clear liquids for a fasting period of at least 6 hours, and administering the preoperative beverage during the fasting period and at least 2 hours before a surgical procedure. The preoperative beverage may include: a carbohydrate providing at least 200 calories; a natural sweetener including monk fruit extract in a quantity ranging between 0.005 and 0.05% by weight; and L-citrulene in a quantity ranging between 0.1 and 0.3% of the preoperative beverage by weight. The preoperative beverage may have an osmolality ranging between 100-300 mOsm. In some cases, the preoperative beverage may have an osmolality ranging between 250-300 mOsm.

In some implementations, the preoperative beverage may also include a combination of various proteins. For example the preoperative beverage may include one or more whole proteins including, for example, any animal and plant-based proteins that may be suitable for use in the preoperative beverage. It may be desirable that the protein or proteins be a clear protein including, for example, a clear whey or a clear pea protein.

In some cases, the preoperative beverage includes one or more of: isoleucine, leucine, valine, lysine, or threonine. In some cases, the preoperative beverage includes an insulinotropic amino. In some cases, the preoperative beverage includes L-glutamine. In some implementations, the preoperative beverage includes caffeine in a quantity ranging between 0.005 and 0.05% of the preoperative beverage by weight and a pH of 3.5 or greater. In some cases, the preoperative beverage may have a pH of 4.0 or greater. In some cases, the preoperative beverage may have a pH of 4.4 or greater. In some cases, the preoperative beverage may have a pH of 4.6 or greater. In some cases, the preoperative beverage may have a pH of 4.8 or greater.

In some implementations, the preoperative beverage includes one or more amino acids in various combinations. For example, the preoperative beverage may include one or more of: L-citruline, L-arginine, L-glutamine, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 depicts an example process for administering a preoperative beverage.

FIG. 2A depicts an example container for a preoperative beverage.

FIG. 2B depicts another example container for a preoperative beverage.

FIG. 2C depicts another example container for a preoperative beverage.

FIG. 2D depicts another example container for a preoperative beverage.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to processes of administering a preoperative beverage and formulations for the preoperative beverage. The preoperative beverage may provide a caloric and nutritional supplement to patients undergoing a surgical operation in which anesthesia is administered. As previously mentioned, traditional guidelines suggest that a patient abstain from solid food and non-clear liquids for a given time period (e.g., fasting period) before a surgical procedure. The fasting period is thought to reduce the risk that the patient's gastric contents will be aspirated while the patient is anesthetized. However, the fasting period prevents the patient from receiving calories and nutrition that may help with post-operative recovery.

Embodiments described herein are directed to a preoperative beverage that may be used to reduce potentially negative consequences of traditional preoperative fasting. In particular, some embodiments described herein are directed to a beverage that provides calories and other nutrients to a patient in a beverage that is administered to a patient 2 hours or longer before a surgical procedure. Providing a source of calories to the patient may reduce insulin resistance and reduce post-surgical trauma experienced by the patient. In addition, a preoperative beverage having sufficient calories may help reduce patient discomfort commonly associated with long periods of fasting. Specifically, patients may be less thirsty, less hungry, and generally more comfortable by receiving a calorie-rich beverage approximately 2-3 hours before surgery.

The beverage may include a minimum of 30 grams of carbohydrate per 50 ml to provide the patient with a sufficient source of calories to improve post-operative recovery and help satisfy a patient's hunger. In addition, it may also be beneficial that the beverage have a relatively high pH to reduce feelings of nausea or sickness in the patient as they come out of anesthesia. As described herein, the beverage may also include one or more vitamins, electrolytes, whole proteins, amino acids, antioxidants, or other nutritional supplements that may be specially formulated to improve recovery and the comfort level of the patient.

However, unlike traditional beverages, like juice, fruit drinks, or sports drinks, there are certain criteria that a preoperative beverage should also satisfy. For example, the osmolality of the beverage must be sufficiently low to ensure that the beverage will be substantially absorbed before the surgical procedure and, more importantly, before the anesthesia is administered. In general, the osmolality of the beverage may be at or below approximately 300 mOsm. In some instances, the osmolality of the beverage falls within a range of approximately 100-300 mOsm. In some instances, the osmolality of the beverage falls within a range of approximately 250-300 mOsm. Thus, a suitable formulation including a source of calories, nutritional supplements, flavoring, and other ingredients should not significantly alter the osmolality of the beverage. In particular, the formulation should not alter the osmolality of the beverage to an extent that the beverage cannot be readily absorbed by the patient's stomach within the 2-hour limit. In some cases, it may be beneficial that the beverage is readily absorbed by the patient's stomach in approximately 90 minutes.

As described herein, it may also be beneficial that the beverage have a pleasant flavor and appearance. In particular, it is desirable to have a beverage that has a natural flavor that is not too sweet or have an unpleasant aftertaste. In some instances, a natural fruit flavoring ingredient is added that, in combination with the carbohydrate calorie source, provides an appropriate flavor. Additionally, the beverage should be free from sediment and not have a cloudy or unusual appearance. The beverage formulations described herein satisfy the strict criteria for osmolality, taste, and appearance, while providing an effective amount of calories and nutritional supplements to aid with patient recovery and comfort.

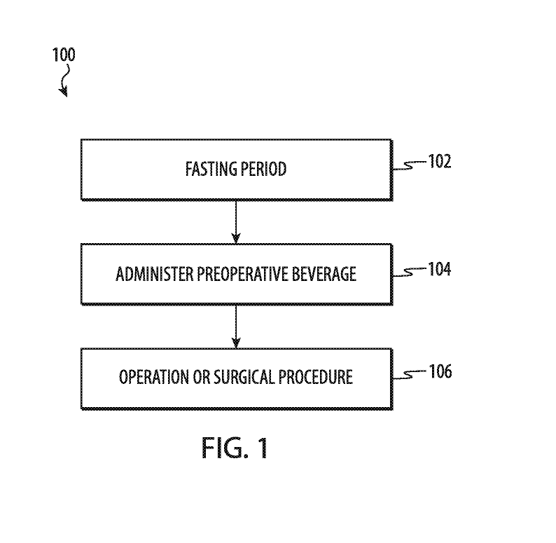

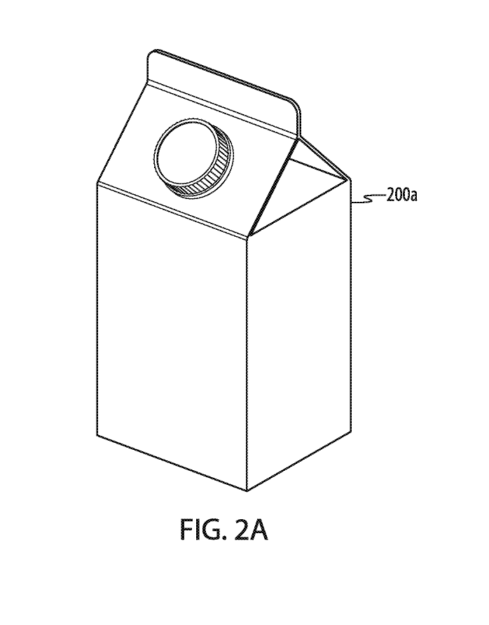

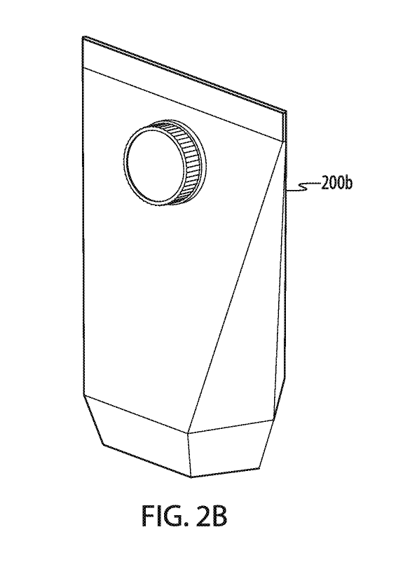

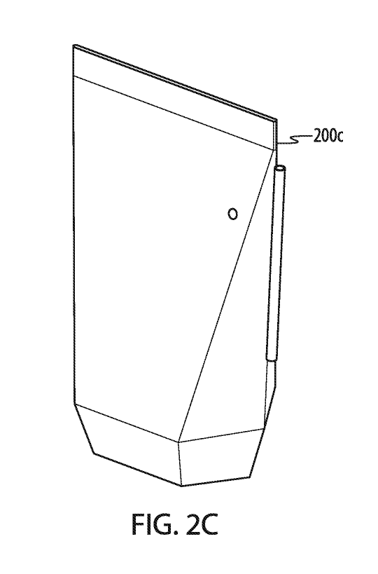

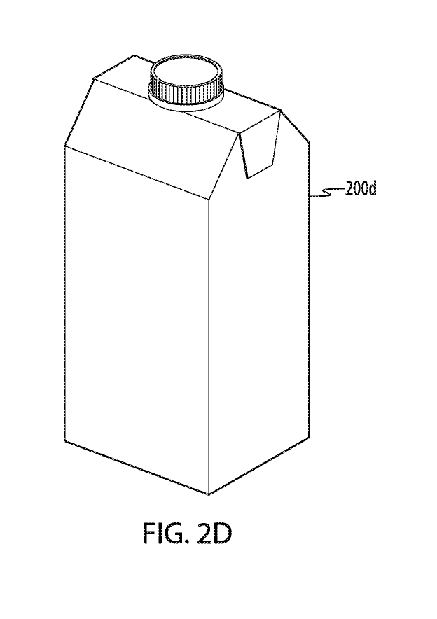

These and other embodiments are discussed below with reference to FIGS. 1-2D. However, those skilled in the art will readily appreciate that the detailed description given herein with respect to these Figures is for explanatory purposes only and should not be construed as limiting.

FIG. 1 depicts an example process 100 for administering a preoperative beverage to a patient. Process 100 may be used to administer the preoperative beverage to the patient before a surgical procedure or operation in which the patient is under a general anesthesia through at least a portion of the procedure. As used herein, general anesthesia may refer to a procedure in which the patient has received medication to place the patient in a temporary and controlled state of unconsciousness. Process 100 is provided as a simplified example and may omit one or more steps or operations that may be performed during a typical beverage administration.

In operation 102, the patient is subjected to a fasting period. In particular, the patient may abstain from ingesting solid foods and non-clear drinks for a given period of time before a scheduled operation or surgical procedure. In some cases, fasting begins at least 6 hours before the scheduled operation or surgical procedure. In some cases, the patient abstains from ingesting any solid food or non-clear beverage after midnight the night before the scheduled operation or surgical procedure. During this time, the patient is generally still allowed to drink a limited amount of clear liquids and take any regular medications (in accordance with accepted guidelines and/or doctor recommendation).

In accordance with some traditional preoperative procedures, the patient will not have a significant source of calories or hydration for many hours before the operation or surgical procedure. As mentioned previously, this fasting period, while recommended under some general guidelines, may cause the patient discomfort and/or inhibit post-operative recovery. Thus, in accordance with process 100, the patient may be given a specially formulated preoperative beverage that may mitigate patient discomfort and also may improve a patient's recovery post-operation.

In operation 104, the preoperative beverage is administered. In particular, a specially formulated preoperative beverage may be administered to a patient during a time period that ranges between 6 hours and 2 hours before an operation or surgical procedure in which the patient may be subjected to general anesthesia. In some cases, the preoperative beverage is administered at least 90 minutes before the operation or surgical procedure. The preoperative beverage may be specially formulated to provide the patient with a source of calories and hydration, while not substantially increasing the risk of perioperative pulmonary aspiration or other complications due to general anesthesia or other surgical procedures. By administering a calorie-rich preoperative beverage, the comfort of the patient may be increased by helping to satiate pre-operative hunger and thirst. The preoperative beverage may also be formulated with a relatively high pH (e.g., having a pH from 4.0 to 4.8 or greater) to help alleviate or reduce nausea before and after the operation or surgical procedure. The preoperative beverage may also provide key nutritional supplements that may aid in patient recovery and increase the overall wellbeing of the patient. Example formulations are discussed in more detail below and may be administered in accordance with operation 104.

With regard to operation 104, it may be beneficial that the preoperative beverage have an osmolality that is compatible with preoperative procedures. Specifically, the preoperative beverage should have an osmolality to help ensure that the patient's stomach is substantially empty before general anesthesia is administered. In some cases, the preoperative beverage has an osmolality within a range of approximately 100-300 mOsm. In some cases, the preoperative beverage has an osmolality within a range of approximately 250-300 mOsm. In general, an osmolality of less than 300 mOsm is suitable for the preoperative beverage. In some cases, the suitability of the preoperative beverage is measured by an estimated time of absorption within the patient's stomach. The preoperative beverage may be absorbed in 2 hours or less. In some cases, the preoperative beverage may be absorbed in 90 minutes or less.

Further in regard to operation 104, the quantity or amount of the preoperative beverage that is administered may also be specially formulated or configured to ensure that the contents of the patient's stomach is substantially empty before the operation or surgical procedure while also providing the patient with calories and other beneficial ingredients. In some cases, the preoperative beverage is administered in a quantity that ranges between 300 ml and 500 ml.

The preoperative beverage may be administered in a single dose or in multiple doses over a given time period. In one example, a single dose of the preoperative beverage having approximately 350 ml of volume by liquid is administered to the patient at least 6 hours before the operation or surgical procedure but not sooner than 2 hours before the operation or surgical procedure. In another example, multiple doses of the preoperative beverage may be administered to the patient up to 2 hours before the operation or surgical procedure. In particular, one or two 350 ml doses of the preoperative beverage are administered the night before surgery and another dose is administered the day of surgery at least 2 hours in advance of the operation or surgical procedure. In another example, multiple doses may be administered, each dose having a volume of less than 350 ml. The total fluid volume of the multiple doses may vary on the timing of the administration, and the quantity of each administration may be reduced over time leading up to the operation or surgical procedure.

In operation 106, the patient undergoes the operation or surgical procedure. After the patient has been administered the preoperative beverage in accordance with operation 104 and an acceptable time period has elapsed, the operation or surgical procedure may be initiated. For example, after at least approximately 2 hours have elapsed since the administration of the preoperative beverage, a general anesthetic may be administered to the patient in preparation for the operation or surgical procedure. In some cases, more than 90 minutes must elapse between administration of the preoperative beverage and the initiation of a general anesthetic procedure.

Process 100 may be used to administer a preoperative beverage in accordance with any of the examples provided herein. That is, any of the example formulations provided in the following disclosure may be administered in a fashion similar to as described above with respect to process 100.

FIGS. 2A-2D depict example packages for storing and administering the preoperative beverage. As shown in FIGS. 2A-2D, the preoperative beverage may be stored in any one of the sterile containers 200a-d. The containers 200a-d may be specially configured to administer the preoperative beverage in a quantity consistent with certain guidelines or directed use. For example, in accordance with a single dose administration scheme, one of the containers 200a-d may contain approximately 360 ml of the preoperative beverage, by volume. In accordance with multiple dose administration schemes, one of the containers 200a-d may include less than 360 ml of the preoperative beverage. In one example, one of the containers 200a-d may include less than 300 ml of the preoperative beverage. Multiple containers 200a-d may be given to the patient up to 2 hours before an operation or surgical procedure.

The containers 200a-d may be filled in accordance with an aseptic packaging process. An aseptic packaging process may provide a shelf life of at least 6 months for the preoperative beverage. In some cases, the shelf life of the preoperative beverage stored in one of the containers 200a-d may be greater than 9 months and in some cases greater than 1 year. In some cases, the shelf life is approximately 12-18 months. The aseptic packaging process and design of the sterile containers 200a-d may also facilitate an extended shelf life for preoperative beverage formulations in which the pH is relatively high (e.g., having a pH of 4.0 to 4.8 or greater). The aseptic packaging process and containers 200a-d may also be formulated to substantially preserve the vitamins, antioxidants, and other nutritional supplements during packaging and the shelf life of the product. The aseptic packaging may also allow for formulations that are shelf stable for 12-18 months without the use of artificial preservatives or other artificial preserving agents.

An aseptic packaging process may include a packaging process in which the container and the preoperative beverage are sterilized or maintained in a sterile environment. Example sterilization processes may include flash heating or other similar techniques. In some cases, the preoperative beverage is subjected to a flash-heating process having a temperature between 85° C. to 150° C. The containers 200a-d may also be subjected to a sterilization process before being filled and may be handled in a sterile environment to reduce the risk of contamination.

In some implementations, the preoperative beverage is packaged using a hot-fill process. For example, the preoperative beverage may be heated to a temperature of 180° C. or greater to sterilize the liquid before it is packaged in a container. In some cases, the preoperative beverage is heated to a temperature of 194° C. or greater before being packaged or inserted into the package. The container may be cooled immediately or soon after being filled, which may aid with sealing and preserving the preoperative beverage.

The containers 200a-d may be formed from a composite or laminate material that is configured to form a barrier to microbiological organisms or other contaminates that may reduce the shelf life of the beverage contents. In some implementations, the containers 200a-d are formed from an outer layer that may include a paper and/or a polymer material. In some cases, the outer layer also includes a metal material, such as aluminum or tin. The outer layer may be bonded or laminated to an inner layer formed from a polymer material, such as polyethylene or a polyamide, or other suitable material. The construction of the containers 200a-d together with an aseptic packaging process may allow the packaged beverage to have a shelf life of greater than 9 months without refrigeration. In some cases, the packaged beverage has a shelf life of greater than 1 year. In some cases, the packaged beverage has a shelf life of greater than 18 months.

The containers 200a-d may also include a mouthpiece or spout that that is adapted for drinking or ingesting the beverage straight from the container. In some cases, the containers 200a-d include a tamper-evident screw cap that is removed and discarded once opened. Since the beverage is intended to be administered over a short period of time, there may not be a need for the cap to be replaceable or re-sealable. In some implementations, the containers 200a-d may include a straw or tube that allows the beverage to be ingested directly from the containers 200a-d.

As previously stated, the preoperative beverage may be specially formulated to both provide the calories and nutrients that a patient may need and also satisfy osmolality requirements to ensure that the beverage is substantially absorbed by the patient's stomach prior to the operation or surgical procedure. Additionally, it may be beneficial that the preoperative beverage have a pleasant flavor and color. The example preoperative beverages described herein have been specially formulated to satisfy these general criteria.

The preoperative beverage is generally a water-based formulation that may include distilled, filtered or purified water. In some instances, the preoperative beverage is formulated using a specialty water including, for example, hydrogen-rich water, H₂ infused water, alkaline water, and other types of specially formulated water.

In general, the preoperative beverage will include a calorie source in a water-based drink. To facilitate rapid absorption by the patient, the calorie source may be primarily provided by a carbohydrate. The carbohydrate may include a glucose, sucrose, and/or fructose type of carbohydrate or sugar. The blend of carbohydrate may be selected based on the calorie requirements as well as the ability to provide a suitable flavor profile. Example carbohydrates include fructose, sucrose, monosaccharides, disaccharides, and polysaccharides, such as maltodextrin, dextrin, and modified starches. The preoperative beverage may include one or more polysaccharides from the mannan family that are derived from plant cell walls including, but not limited, to glucomannans and acemannan.

The carbohydrate may be added in a quantity of approximately 30 g or greater per 50 ml of volume liquid in order to produce a preoperative beverage having approximately 150 calories or greater. In some instances, the carbohydrate may be added in approximately 40 g or greater per 50 ml of volume liquid. In some instances, the carbohydrate may be added in approximately 50 g or greater per 50 ml of volume liquid to produce a preoperative beverage having approximately 200 calories or greater.

In some beverage formulations, a polysaccharide is used to provide the primary source of calories for the beverage and is blended with glucose and fructose to provide the desired flavor while still meeting the necessary criteria for osmolality. In some cases, a natural sweetener may be used to balance the flavor of the polysaccharide while also providing a beverage that is readily absorbed by the patient. Example natural sweeteners include fruit juice or fruit concentrate and may include ingredients made from organic or organically produced fruit products. The natural sweetener may include monk fruit concentrate, stevia extract, or other similar sweeteners derived from natural or organic products.

The preoperative beverage may also have a pH level that is specially formulated to reduce nausea of the patient before and after anesthesia has been administered. A preoperative beverage having a higher pH may be considered a low acid drink and may help calm the patient's stomach. In some instances, the pH of the preoperative beverage is 3.5 or greater. In some instances, the pH of the preoperative beverage is 4.0 or greater. In some instances, the pH of the preoperative beverage is 4.3 or greater. In some instances, the pH of the preoperative beverage is 4.5 or greater. In some instances, the pH of the preoperative beverage is 4.6 or greater. In some instances, the pH of the preoperative beverage is 4.8 or greater. The pH of the formulation may be adjusted through the addition of various ingredients like citric acid, ascorbic acid, or other similar ingredients. The pH may also be adjusted with the addition of proteins, amino acids, caffeine, or other pH-altering ingredients. By increasing the pH, patient discomfort may be decreased and the flavor of the formulation may be enhanced. However, a preoperative beverage having a pH of 4.0 or greater may be packaged using an aseptic packaging process in order to achieve an acceptable shelf life. In some instances, the preoperative beverage may be packaged using a hot-fill process, which may allow for lower pH levels.

Provided that the preoperative beverage maintains the required levels of osmolality, other nutrients may be added to the formulation. The other nutrients may be configured to facilitate patient recovery, healing, and general patient wellbeing. In particular, the preoperative beverage may include certain amino acids alone or in combination with certain vitamins to help improve patient recovery and/or overall patient wellbeing. For example, a formulation of the preoperative beverage that includes certain amino acids may reduce muscle breakdown and support circulatory and skeletal muscle health. Certain amino acids may also increase circulating plasma prealbumin and retinol-binding proteins. The addition of amino acids may also stimulate insulin secretion, which may be particularly beneficial during or before periods of fasting.

In some implementations, the preoperative beverage includes L-citruline in effective quantities. L-citruline is an amino acid that may be converted to arginine by the kidneys. The addition of effective amounts of L-citruline may increase circulation and nitric oxide, which may be helpful in patients recovering from most types of surgical procedures. L-citruline may also aid in lowering a patient's blood pressure. For a 360 ml dose of the preoperative beverage, a quantity of 3-5 g of L-citruline may be an effective quantity. Other quantities of L-citruline may also be effective. In some cases, the preoperative beverage may include 0.1 to 0.5% L-citruline by weight. In some cases, the preoperative beverage includes between 0.15 and 0.3% L-citruline by weight. The preoperative beverage may also include other similar amino acids, such as, L-arginine, which may produce similar results.

The preoperative beverage may also include other amino acids, including, for example, insulinogenic BCAA amino acids. Example amino acids include isoleucine, leucine, valine, lysine, and threonine. In general, BCCA amino acids are not naturally synthesized by the body. By including BCCA amino acids, the beverage may be catabolized by extrahepatic tissues to enhance protein synthesis. The addition of BCCA amino acids may also inhibit muscle breakdown and provide other health benefits, such as decreasing the patient's body stress index, increasing circulating plasma prealbumin, increasing retinol-binding proteins, and/or stimulating insulin secretion. In some implementations, the preoperative beverage may include insulinotropic amino acids and other amino acids that support circulatory and skeletal muscle health. The preoperative beverage may also include effective quantities of L-glutamine to reduce or minimize the breakdown of muscle.

In some implementations, the preoperative beverage includes one or more amino acids in various combinations. For example, the preoperative beverage may include one or more of: L-citruline, L-arginine, L-glutamine, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The addition of amino acids may also have synergistic effects with the addition of certain vitamin supplements. For example, L-citruline in combination with vitamin C and/or vitamin D may produce certain benefits for the patient. For example, L-citruline in combination with vitamin C may facilitate endothelial nitric oxide production to promote cardiovascular health. The addition of a vitamin D supplement may also be beneficial as low levels of vitamin D may reduce the bioavailability of nitric oxide in veins and arteries. The combination of vitamin C with L-citruline may also help support increased levels of L-citruline, which may enhance the health benefits to the patient.

The preoperative beverage may also include a combination of various proteins. For example, the preoperative beverage may include one or more whole proteins. Example whole proteins include any animal and plant-based proteins that may be suitable for use in the preoperative beverage. It may be desirable that the protein(s) be a clear protein including, for example, a clear whey or a clear pea protein. In general, it may be beneficial that the protein not adversely affect taste of the preoperative beverage. In some cases, the flavor additives are adjusted to help balance any effects on taste due to the addition of a protein. It may also be beneficial that the protein not adversely affect the osmolality or adversely affect rate of absorption of the beverage in the stomach of the patient.

The preoperative beverage may also include a combination of vitamin supplements that are selected to provide the patient with additional benefits during post-operation recovery. Specifically, the preoperative beverage may include effective quantities of vitamin A, which may supplement vitamin deficiencies caused by fasting. Effective quantities of vitamin A may also aid in wound healing and rebuilding of soft tissue. The preoperative beverage may also include effective quantities of vitamin C, which may also aid in wound healing. The preoperative beverage may also include effective quantities of vitamin D and other vitamins that may promote the general health of the patient. In some embodiments, the preoperative beverage also includes one or more antioxidants including, for example, resveratrol, lactulose, or epicatechins.

Importantly, the addition of amino acids alone or in combination with vitamin supplements should not adversely affect the osmolality of the preoperative beverage to the point in which it may not be suitable for use as a preoperative beverage. Specifically, the preoperative beverage including the calorie source (e.g., carbohydrates), natural flavorings, whole proteins, amino acids, vitamins, antioxidants, or other nutritional supplements, as a formulation has an osmolality less than 300 mOsm and may fall within a range of approximately 100-300 mOsm.

Some beverage formulations may also include electrolytes or salts to help rehydrate the patient. For example, the preoperative beverage may include effective quantities of sodium (e.g., sodium chloride) and/or potassium to replace or supplement electrolytes lost during the operation or during postoperative recovery. In some implementations, the preoperative beverage may include effective quantities of other electrolytes including, for example, magnesium, phosphorous, and/or calcium.

The preoperative beverage may also include stimulants, which may improve the patient's mental state, increase blood flow, and provide other benefits during post-operative recovery. In particular, the preoperative beverage may include caffeine in amounts that provide effective results for the patient, but also do not adversely affect the osmolality of the beverage. Additionally, the amount of caffeine should not adversely affect the pH level of the beverage or the flavor that has been formulated with a particular selection of carbohydrates and natural flavorings. In some implementations, the preoperative beverage includes 20 mg or more of caffeine per 50 g of beverage. In some cases, the preoperative beverage may include 40 mg or more of caffeine per 50 g of beverage. In some cases, the preoperative beverage includes 60 mg or more of caffeine per 50 g of beverage. In some cases, the preoperative beverage includes less than 80 mg of caffeine per 50 g of beverage.

The following formulations are provided as example beverage formulations that are suitable for use as a preoperative calorie supplement. These examples are provided as specific implementations that satisfy the criteria. In practice, the specific ingredients and their relative quantities may vary to a certain degree while still being suitable for use.

### Example Formulation 1

In one example formulation, the preoperative beverage may be a water-based product that is specially configured to provide a source of calories to the patient in a high pH and enriched formulation that provides supplements to a patient that may be particularly beneficial during post-operative recovery. Specifically, in the present example, the preoperative beverage provides at least 200 calories in a water-based beverage having an osmolality ranging between 250-300 mOsm to facilitate absorption within 1-2 hours. In some cases, the osmolality ranges between 100-300 mOsm. In the present example, the preoperative beverage also includes a pH of 4.0-4.6 or greater. In some cases, the pH ranges between 4.3 and 4.6. When packaged using an aseptic packaging process and container, as described in the examples above, the preoperative beverage should be shelf stable for a period of 12-18 months. The preoperative beverage may be stored in an aseptic container having a 150-360 ml dose.

The present example is also formulated to provide a natural flavor without the use of artificial flavoring agents or sweeteners. The preoperative beverage of the present example has also been formulated to be clear or at least substantially free of sediment.

The preoperative beverage of the present example may include the following ingredients in approximately the following ranges. The beverage may include a water base forming approximately 80-85% of the formulation by weight. The preoperative beverage may include a specialty water including, for example, hydrogen-rich water, H₂ infused water, alkaline water, and other types of specially formulated water.

The beverage may also include Maltodextrin M100 in a quantity ranging between 10-15% of the formulation by weight. The beverage may also include crystalline fructose in a quantity ranging between 1-3% by weight.

The beverage also includes L-citruline in a quantity ranging between 0.1 and 0.3% by weight. The beverage also includes vitamin A Palmitate in a quantity ranging between 0.0005 and 0.002% by weight. The beverage also includes vitamin D3 in a quantity ranging between 0.0005 and 0.002% by weight. The beverage also includes sodium chloride in a quantity ranging between 0.02 and 0.005% by weight. The beverage also includes potassium citrate monohydrate in a quantity ranging between 0.03 and 0.1% by weight. The beverage also includes monk fruit extract in a quantity ranging between 0.005 and 0.05% by weight. The beverage also includes ascorbic acid in a quantity ranging between 0.005 and 0.002% by weight. The beverage also includes a natural flavoring agent in a quantity ranging between 0.2 and 1.5% by weight.

### Example Formulation 2

In one example formulation, the preoperative beverage may be a water-based product that is specially configured to provide a source of calories to the patient in a high pH and enriched formulation that provides supplements to a patient that may be particularly beneficial during post-operative recovery. Specifically, in the present example, the preoperative beverage provides at least 200 calories in a water-based beverage having an osmolality ranging between 250-300 mOsm to facilitate absorption within 1-2 hours. In some cases, the osmolality ranges between 100-300 mOsm.

In the present example, the preoperative beverage may also include a stimulant, such as caffeine, to improve the mental state of the patient, improve blood flow, and provide other beneficial effects for the patient.

In the present example, the preoperative beverage also includes a pH of 4.0-4.6 or greater. In some cases, the pH ranges between 4.3 and 4.6. When packaged using an aseptic packaging process and container, as described in the examples above, the preoperative beverage should be shelf stable for a period of 12-18 months. The preoperative beverage may be stored in an aseptic container having a 150-360 ml dose.

The present example is also formulated to provide a natural flavor without the use of artificial flavoring agents or sweeteners. The preoperative beverage of the present example has also been formulated to be clear or at least substantially free of sediment.

The preoperative beverage of the present example may include the following ingredients in approximately the following ranges. The beverage may include a water base forming approximately 80-85% of the formulation by weight. The preoperative beverage may include a specialty water including, for example, hydrogen-rich water, H₂ infused water, alkaline water, and other types of specially formulated water. The beverage may also include Maltodextrin M100 in a quantity ranging between 10-15% of the formulation by weight. The beverage may also include crystalline fructose in a quantity ranging between 1-3% by weight.

The beverage may also include natural caffeine in a quantity ranging between 0.005 and 0.05% by weight. In some cases, the natural caffeine is added in a quantity ranging between 0.01 and 0.04% by weight.

The beverage also includes L-citruline in a quantity ranging between 0.1 and 0.3% by weight. The beverage also includes vitamin A Palmitate in a quantity ranging between 0.0005 and 0.002% by weight. The beverage also includes vitamin D3 in a quantity ranging between 0.0005 and 0.002% by weight. The beverage also includes sodium chloride in a quantity ranging between 0.02 and 0.005% by weight. The beverage also includes potassium citrate monohydrate in a quantity ranging between 0.03 and 0.1% by weight. The beverage also includes monk fruit extract in a quantity ranging between 0.005 and 0.05% by weight. The beverage also includes ascorbic acid in a quantity ranging between 0.005 and 0.002% by weight. The beverage also includes a natural flavoring agent in a quantity ranging between 0.2 and 1.5% by weight.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method of administering a preoperative beverage to a patient, the method comprising:
fasting from solid foods for a fasting period of at least 6 hours;
administering the preoperative beverage during the fasting period and at least 2 hours before a surgical procedure, wherein the preoperative beverage comprises:
a water-based solution having a pH of 4.3 or greater;
a carbohydrate including maltodextrin in a quantity ranging between 10-15% of the preoperative beverage by weight; and
an amino acid in a quantity ranging between 0.1 and 0.3% of the preoperative beverage by weight; wherein:
the preoperative beverage has an osmolality ranging between 100-300 mOsm.

2. The method of administering the preoperative beverage of claim 1, wherein:
the preoperative beverage has an osmolality ranging between 250-300 mOsm; and
the preoperative beverage is configured to be absorbed by a patient's stomach within approximately 90 minutes from ingestion.

3. The method of administering the preoperative beverage of claim 1, wherein the preoperative beverage further comprises:
vitamin A in a quantity ranging between 0.0005 and 0.002% of the preoperative beverage by weight; and
vitamin D3 in a quantity ranging between 0.0005 and 0.002% of the preoperative beverage by weight.

4. The method of administering the preoperative beverage of claim 1, wherein the preoperative beverage further comprises caffeine in a quantity ranging between 0.005 and 0.05% of the preoperative beverage by weight.

5. The method of administering the preoperative beverage of claim 1, wherein the preoperative beverage further comprises a natural sweetener including monk fruit extract in a quantity ranging between 0.005 and 0.05% by weight.

6. The method of administering the preoperative beverage of claim 1, wherein the preoperative beverage is shelf stable for a period of at least 12 months when packaged.

7. The method of administering the preoperative beverage of claim 1, wherein the preoperative beverage is aseptically packaged in an aseptic container.

8. A preoperative beverage for administration between 2 to 6 hours before a surgical procedure, the preoperative beverage comprising:
a water-based solution having a pH of 4.3 or greater;
a carbohydrate including maltodextrin in a quantity ranging between 10-15% of the preoperative beverage by weight; and
an amino acid including L-citruline in a quantity ranging between 0.1 and 0.3% of the preoperative beverage by weight; wherein:
the preoperative beverage has an osmolality ranging between 100-300 mOsm.

9. The preoperative beverage of claim 8, wherein the preoperative beverage is shelf stable for a period of at least 12 months when packaged.

10. The preoperative beverage of claim 8, wherein the preoperative beverage is packaged using an aseptic container.

11. The preoperative beverage of claim 8, further comprising:
vitamin A in a quantity ranging between 0.0005 and 0.002% of the preoperative beverage by weight; and
vitamin D3 in a quantity ranging between 0.0005 and 0.002% of the preoperative beverage by weight.

12. The preoperative beverage of claim 8, further comprising caffeine in a quantity ranging between 0.005 and 0.05% of the preoperative beverage by weight.

13. The preoperative beverage of claim 8, further comprising a natural sweetener including monk fruit extract in a quantity ranging between 0.005 and 0.05% by weight;

14. A method of administering a preoperative beverage, comprising:
abstaining from solid foods and non-clear liquids for a fasting period of at least 6 hours;
administering the preoperative beverage during the fasting period and at least 2 hours before a surgical procedure, wherein the preoperative beverage comprises:
a carbohydrate providing at least 200 calories;
a natural sweetener including monk fruit extract in a quantity ranging between 0.005 and 0.05% by weight; and
L-citrulene in a quantity ranging between 0.1 and 0.3% of the preoperative beverage by weight; wherein:
the preoperative beverage has an osmolality ranging between 100-300 mOsm.

15. The method of administering the preoperative beverage of claim 14, wherein the preoperative beverage further comprises one or more of:
isoleucine, leucine, valine, lysine, or threonine.

16. The method of administering the preoperative beverage of claim 14, wherein the preoperative beverage further comprises an insulinotropic amino.

17. The method of administering the preoperative beverage of claim 14, wherein the preoperative beverage further comprises L-glutamine.

18. The method of administering the preoperative beverage of claim 14, wherein the preoperative beverage further comprises caffeine in a quantity ranging between 0.005 and 0.05% of the preoperative beverage by weight.

19. The method of administering the preoperative beverage of claim 14, wherein the preoperative beverage further comprises vitamin A in a quantity ranging between 0.0005 and 0.002% of the preoperative beverage by weight.

20. The method of administering the preoperative beverage of claim 14, wherein the preoperative beverage further comprises vitamin D3 in a quantity ranging between 0.0005 and 0.002% of the preoperative beverage by weight.
